(19) United States
(12) Patent Application Publication
(10) Pub. No.: US 2016/0313313 A1
(43) Pub. Date: Oct. 27, 2016

(54) LATERAL FLOW ASSAY APPARATUS AND METHOD, AND SENSOR THERFOR

(71) Applicant: HER MAJESTY THE QUEEN IN RIGHT OF CANADA, as represented by THE MINISTER OF NATIONAL DEFENCE, Ottawa (CA)

(72) Inventors: Ryan J. Love, Burlington (CA); Kristin Topping, Kingston (CA); Michel A. Paul, Toronto (CA); Heinz-Bernhard Kraatz, Pickering (CA)

(21) Appl. No.: 15/101,007

(22) PCT Filed: Jan. 16, 2014
(86) PCT No.: PCT/CA2014/000027

§ 371 Date: Jun. 2, 2016

(51) Int. Cl. G01N 33/543; G01N 27/327; G01N 33/74

(52) U.S. Cl. CPC G01N 33/5438; G01N 33/74; G01N 27/3276; G01N 2333/575

(57) ABSTRACT

An apparatus for electrochemically detecting and/or measuring an analyte (e.g. melatonin) in a fluid sample has a dry porous carrier that permits flow of a fluid sample by capillary action to biosensor (e.g. an analyte detection electrode set). The biosensor has a working electrode and a counter electrode disposed across the carrier in a non-parallel direction in relation to a direction of fluid flow such that the fluid sample crosses at least the working electrode embedded in the carrier. The working electrode has an analyte-specific binder immobilized on an electrically conductive material and the biosensor is configured to electrochemically, e.g. voltammetrically, detect the presence of and/or measure the amount of the analyte bound at the working electrode after the fluid sample crosses the working electrode.

### FIELD

This application relates to methods and apparatuses for detecting analytes in a fluid.

### BACKGROUND

A highly sensitive and specific device for rapid detection and quantification of small analytes is desirable for a number of different reasons. Several endocrine and neuroendocrine biomarkers for common ailments exist including, but not limited to: (1) fatigue and circadian rhythm misalignment (melatonin); (2) excessive stress (cortisol), which may lead to post-traumatic stress disorder; and (3) chronic overexertion (cortisol, androgens, and estrogens). Rapid detection of such analytes in a biological fluid (e.g. saliva, urine or blood serum) would greatly hasten detection of such ailments, lead to more rapid recovery, and greatly improve the health and wellness of people. Furthermore, a specific device tailored to pathogen detection could be used to test water collected from suspect sources or concentrated air samples for viral, bacterial or fungal contaminants.

The detection and quantification of small analytes currently requires extensive laboratory work. For example, quantification of small-molecule hormones, such as melatonin, serotonin, androgens, estrogens and cortisol, is generally performed by plate-based enzyme immunoassays (EIA). The EIA requires highly-qualified personnel to properly run the assay using expensive and complicated laboratory equipment, and requires 24 to 36 hours to complete. Reliable detection and quantification of biological pathogens similarly require extensive laboratory work by properly trained technicians. A variety of field deployable, point detection technologies currently exist for well-defined and characterized biological threats. These technologies include hand-held immunoassay test kits, fluorescent aerosol particle sizing and polymerase-chain reaction. Each of these detection techniques has an inherent disadvantage including, but not limited to, being difficult to use, having an exorbitant cost and time requirements, a high false positive rate or pathogen library limitations, i.e., the detection of novel agents is virtually impossible.

Primary limitations of the current technology for quantification of small biological analytes are (1) the time required to perform the assay, up to 36 hours; and (2) the bulky and expensive equipment that is required. Furthermore, radioactive probes are required for increased sensitivity of such assays, which increases the precautions that must be taken when performing the procedure. The financial expense of current commercially available technology is also a limitation as a kit for the analysis of 40 samples costs $400 to $1000 depending on the analyte. Most importantly, current technology does not permit rapid self-assessment of small analytes in the field, which is required for diagnostic hormone assessment and detection of biological threats.

A biosensor for salivary melatonin has been desired by the chronobiology research community for over the past two decades; however the necessary specifications of such a sensor are such that many challenges have to be overcome. First, the sensor must be able to detect dim light melatonin onset (OLMO), which means that the minimum detection sensitivity of the sensor should be about 10 μg/ml. Second, with regards to the specificity, the sensor must be able to detect melatonin without interference from tryptophan, or tryptophan-derived endocrines such as serotonin.

There remains a need for rapid detection and quantification of small analytes, for example melatonin, that overcomes one or more of the limitations of current technologies.

### SUMMARY

There is provided an apparatus for electrochemically detecting and/or measuring an analyte in a fluid sample, the apparatus comprising: a dry porous carrier that permits flow of a fluid sample from a first portion of the carrier to a second portion of the carrier; and, an analyte detection electrode set comprising a working electrode and a counter electrode disposed across the carrier in a non-parallel direction in relation to a direction of fluid flow such that the fluid sample flowing from the first portion of the carrier to the second portion of the carrier crosses at least the working electrode, at least the working electrode embedded in the carrier, the working electrode comprising an analyte-specific binder immobilized on an electrically conductive material, the analyte detection electrode set configured to electrochemically detect a presence of and/or measure an amount of the analyte bound to the analyte-specific binder immobilized on the working electrode after the fluid sample crosses the working electrode.

There is further provided a biosensor for detecting and/or measuring melatonin in a fluid sample, the biosensor comprising an electrochemical device comprising: a working electrode with a self-assembled monolayer of a melatonin-specific antibody immobilized thereon, the antibody attached to the working electrode by a bifunctional linker; and, a counter electrode.

There is further provided a method of electrochemically detecting and/or measuring an analyte in a fluid sample, the method comprising: contacting a fluid sample with a dry porous carrier to permit flow of the fluid sample in the carrier by capillary action; permitting the fluid sample to contact an analyte detection electrode set comprising a working electrode and a counter electrode embedded in the carrier, the working electrode comprising an analyte-specific binder immobilized on an electrically conductive material, the analyte-specific binder binding analyte present in the fluid sample thereby immobilizing the analyte at the working electrode; and, permitting the fluid sample to flow past the analyte detection electrode set, detecting the fluid sample with a fluid detection electrode set after the fluid sample has passed the analyte detection electrode set and initiating electrochemical detection and/or measurement of the analyte by the analyte detection electrode set when the fluid detection electrode set detects the fluid sample.

It has now been found that rapid electrochemical, especially voltammetric, detection and/or measurement of an analyte in a fluid sample may be attained with an apparatus in which a detecting electrode set having an analyte-specific binder immobilized thereon is embedded in a porous carrier, the carrier permitting the fluid sample to flow by capillary action to contact the detecting electrode set. The invention is particularly well suited for the detection and/or measurement of hormones or endocrines, especially small-molecule endocrines, for example, melatonin, serotonin, cortisol, androgens and estrogens, especially melatonin. The fluid sample may be any liquid containing an analyte of interest, preferably an aqueous liquid. Aqueous liquids include, for example, environmental water samples or biological fluids. Biological fluids are of particular interest and include, for example saliva, blood or urine, especially saliva. As a result, endocrine sensors are provided herein, which are capable of quantifying physical/psychological stress, androgenic potential and overtaining, and detection of a range of endocrine-related pathologies (e.g. adrenal insufficiency) in a subject, especially a human subject.

The present invention provides a portable, highly sensitive, fully quantitative lateral flow immunoassay for small analytes with voltammetry signal output. This invention permits rapid quantification of small analytes in a wide range of fluids. Applications include, for example, salivary endocrine analysis to determine a person's endogenous hormone concentrations, detection of pathogens or chemical contaminants in a water source and detection of chemical warfare agents in air (by testing the fluid formed from condensed air). Endocrine analysis may be used to enhance a person's performance through optimization of androgen concentrations or for early detection of dangerous hormone imbalances that cause depression and post-traumatic stress disorder. Self-assessment of an analyte rapidly and in any environment may be performed and cost associated with individual sample analysis is much lower than the current commercially available technology.

In a particularly preferred embodiment, the invention permits rapid quantification of salivary melatonin concentration. As a biomarker of fatigue, salivary melatonin analysis may be used widely by transport operators (truck, train, aircraft, etc.) or regulations officers to reduce the incidence of fatigue related accidents. Furthermore, this invention could be used to optimize individual performance by assisting with the adjustment of circadian rhythm to a desired schedule.

Further features will be described or will become apparent in the course of the following detailed description. It should be understood that each feature described herein may be utilized in any combination with any one or more of the other described features, and that each feature does not necessarily rely on the presence of another feature except where evident to one of skill in the art.

### BRIEF DESCRIPTION OF THE DRAWINGS

For clearer understanding, preferred embodiments will now be described in detail by way of example, with reference to the accompanying drawings, in which:

FIG. 1 depicts a lateral flow assay device design in accordance with the present invention in which Assay Stage 1 comprises a sample introduction pad, Assay Stage 2 comprises a fibrous segment, Assay Stage 3 comprises a nitrocellulose support segment including a three electrode test wire set embedded in the nitrocellulose, and Assay Stage 4 comprises a run-off pad.

FIG. 2 is a block diagram depicting a control circuit for the lateral flow assay device of FIG. 1

FIG. 3 depicts an illustration of a self-assembled monolayer (SAM) formed on a gold electrode surface to permit selective detection of melatonin.

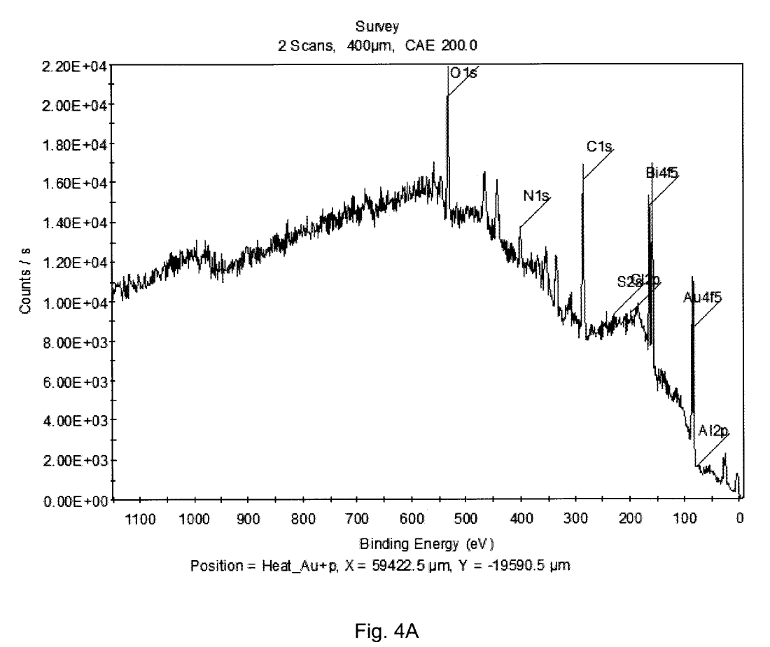

FIG. 4 depicts X-ray photoelectron spectroscopy characterization of a gold working electrode after critical SAM assembly steps. FIG. 4A depicts XPS scan of a lipoic acid SAM attached to the gold working electrode. FIG. 4B depicts XPS scan of an antibody SAM attached to lipoic acid on the gold working electrode.

FIG. 5 depicts cyclic voltammograms (CV) of bare and surface-modified electrodes. FIG. 5A illustrates that formation of the SAM of lipoic acid reduced the Faradaic redox currents that can be seen from the oxidation/reduction of Fe[CN]₆)^{3-/4-} on bare gold. For FIG. 5B, an anti-melatonin antibody was anchored on the surface by reacting selectively with the N-hydroxysuccinimide on lipoic acid, which caused a further decrease in the redox current. To block the non-specific adsorption of proteins and other species to the surface of the electrode, the surface was treated with pooled human saliva (melatonin-free), which reduced redox currents even further by blocking direct access of the conducting ions to the electrode surface.

FIG. 6 depicts a graph of potential (V) vs. current density (μA/cm²) after square-wave voltammetry (SWV) experiments using aqueous melatonin solutions of known concentration. The plot of melatonin concentration (ng/ml) vs. peak current (μA/cm²) clearly shows a linear relationship between melatonin and current produced by the SWV experiments.

FIG. 7 depicts a graph of potential (V) vs. current (nA) from SWV experiments performed in redox probed after electrodes were incubated for 1 hour in saliva with a known concentration of melatonin. The plot of melatonin concentration (μg/ml) vs. peak current (nA) clearly shows a linear relationship with a correlation coefficient of 0.9936.

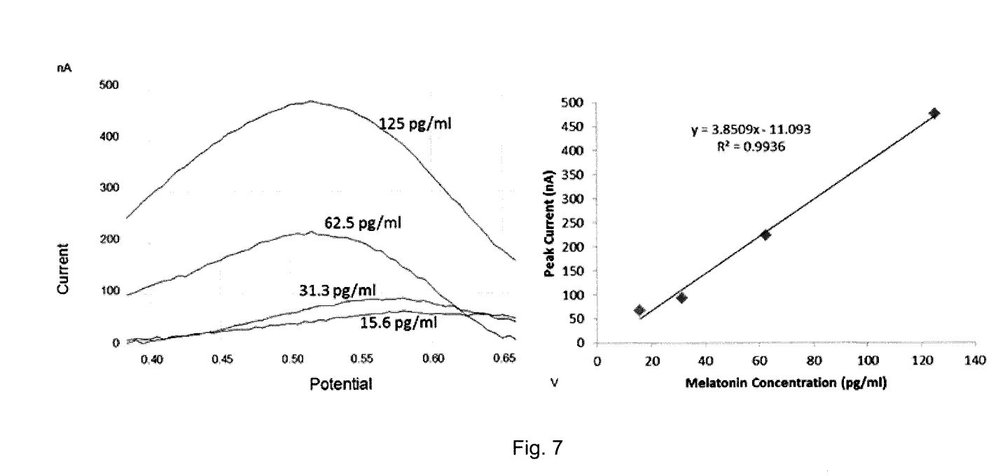

FIG. 8A and FIG. 8B depict voltammograms from a Testing Electrode Set in a device designed in accordance with the present invention showing the detection of ferrocyanide oxidation. FIG. 8A depicts a linear voltammogram with Faradaic current (from ferrocyanide oxidation) on top of non-Faradaic current caused by charge transfer to the electrolyte (sodium perchlorate). FIG. 8B depicts a linear voltammogram showing only the current corresponding to ferrocyanide oxidation.

### DETAILED DESCRIPTION

The analyte detection electrode set functions to electrochemically detect and/or measure the analyte. Electrochemical detection and/or measurement may be accomplished by any suitable electroanalytical technique; however, it is preferable to use voltammetry whereby information about the analyte is obtained by measuring current as potential is varied. Voltammetry may include, for example, linear sweep voltammetry, staircase voltammetry, square-wave voltammetry, cyclic voltammetry, anodic stripping voltammetry, cathodic stripping voltammetry, adsorptive stripping voltammetry, normal pulse voltammetry or differential pulse voltammetry. Cyclic voltammetry is particularly preferred. The apparatus may be pre-set with a pre-determined electrochemical protocol that may be particularly well suited for the particular analyte of interest.

The analyte detection electrode set comprises a working electrode and a counter electrode, and may further comprise a reference electrode if desired. The electrodes are disposed across the carrier in a non-parallel direction in relation to a direction of fluid flow such that the fluid sample must flow at least across the working electrode, and preferably across all of the electrodes. Preferably the electrodes extend across the entire width of the carrier so that there is less chance that the fluid sample will miss the electrodes and also to provide more working electrode surface to which the analyte can bind. The width of the carrier may be defined as the dimension perpendicular to the direction of fluid flow. Preferably the electrodes are arranged to be perpendicular to the direction of fluid flow. The electrodes may be provided as thin wires or thin and narrow strips.

It is particularly noteworthy that at least the working electrode is embedded in the carrier, and preferably all of the electrodes are embedded. Embedding the electrodes in the carrier protects the electrodes from physical and/or chemical damage and protects the analyte-specific binder from contacting spurious analyte in the environment prior to utilizing the apparatus. Embedding the electrodes also provides a stable, protected environment for conducting the electrochemical procedure used for detecting the analyte. The embedded electrodes permits the passage of fluid over each electrode and ensures that any analyte dispersed in the fluid must pass over the analyte-specific binder that is immobilized on the electrode surface.

The electrodes comprise electrically conductive materials. Electrodes may be electrically conductive metals, electrically conductive non-metals or mixtures thereof. Metals include, for example, gold, silver, copper, platinum, palladium, rhodium, tungsten or alloys thereof. Non-metals include, for example, metal oxides, electrically conductive carbon (e.g. graphite, carbon nanotubes), doped silicon oxides and the like. Preferably, the electrodes comprise metals, more preferably gold, platinum or silver. The working electrode preferably comprises gold. The counter electrode preferably comprises platinum. The reference electrode preferably comprises silver. The electrodes may be formed as free-standing structures or may be printed on to a substrate, for example the electrodes may be printed on the carrier. The electrodes may be sandwiched between two pieces of the carrier and then the pieces of the carrier sealed together (e.g. with an adhesive) to ensure that the electrodes are embedded in the carrier.

The working electrode comprises an analyte-specific binder immobilized on the electrically conductive material. The analyte-specific binder specifically binds the analyte of interest to the exclusion of other chemical species in the fluid sample, and preferably to the exclusion of all other chemical species. The analyte-specific binder is preferably a protein, more preferably an antibody specific to the analyte. A peptide with a specific affinity for the analyte may also be suitable, for example the extracellular fragment of a cellular receptor that is specific for the analyte. Antibodies may be monoclonal or polyclonal, preferably polyclonal. For example, the analyte-specific binder may be specific for melatonin, preferably a melatonin-specific antibody, more preferably a melatonin-specific polyclonal antibody.

Immobilizing the analyte-specific binder on the electrically conductive material is done to prevent the analyte-specific binder from diffusing away from the electrically conductive material when the fluid sample passes the working electrode. In order to conduct the electrochemical detection and/or measurement at the electrodes, the analyte bound to the analyte-specific binder must remain at the electrodes. The analyte-specific binder is preferably covalently bound to the electrically conductive material. The analyte-specific binder is preferably bound to the surface of electrically conductive material. The analyte-specific binder may be attached directly to the electrically conductive material, but in many instances this is not possible, therefore, the analyte-specific binder is preferably attached through a linker. The linker may be a bifunctional linker that is capable of binding to the electrically conductive material and to the analyte-specific binder while leaving the analyte-specific binder capable of binding to the analyte. Suitable linkers include lipoates, for example, N-hydroxysuccinimide esters containing a mercapto or thiol group (e.g. dihydrolipoic acid N-hydroxysuccinimide ester, 12-mercaptododecanoic acid N-hydroxysuccinimide ester, 3,3′-dithiodipropionic acid di(N-hydroxysuccinimide ester) and N-succinimidyl-3-thiopropionate.

The analyte-specific binder preferably forms a self-assembled monolayer (SAM) on the surface of the electrically conductive material. SAMs increase the exposed surface area of analyte-specific binder increasing the number of sites to which the analyte may bind thereby increasing the amount of analyte that may be captured, while still providing good electron flow between the working electrode and the analyte to permit the redox reaction to occur at the analyte. Where a linker is used to link the analyte-specific binder to the electrically conductive material, the linker may form a SAM on the electrically conductive material, and linking the analyte-specific binder to the linker thereby creates a SAM of the analyte-specific binder on the electrically conductive material.

The formation of a SAM on the electrically conductive material may leave gaps that expose the electrically conductive material. Such gaps may be back-filled with an analyte-free fluid. The analyte-free fluid may comprise, for example, analyte-free saliva (e.g. human pooled saliva) or polyethylene glycol. Where the fluid sample is saliva, the analyte-free fluid used for back-filling preferably comprises analyte-free saliva.

The analyte detection electrode set is an electrochemical device that may be viewed as part of a sensor. When the analyte-specific binder comprises a biological molecule (e.g. a protein or an antibody), the sensor is a biosensor. When the analyte-specific binder comprises an antibody, the sensor is an immunobiosensor. The sensor may be capable of directly determining the concentration of the analyte in the fluid sample because the SAM of the analyte-specific binder on the working electrode surface binds sufficient analyte that electric current resulting from analyte oxidation (or reduction) correlates well with analyte concentration.

The dry porous carrier comprises a first portion before the analyte detection electrode set and a second portion after the analyte detection electrode set. The fluid sample permeates through the carrier from the first portion to the second portion, and in so doing passes the analyte detection electrode set (i.e. the sensor) where analyte in the fluid sample becomes bound to the analyte-specific binder and may be detected and/or measured.

The first portion of the carrier may comprise a fluid sample introduction portion that permits introduction of the fluid sample on to the carrier, for example in the form of a droplet. The second portion of the carrier may comprise a run-off portion that permits the sample fluid to continue flowing past the analyte detection electrode set by capillary action. The analyte detection electrode set is located between the fluid sample introduction portion and the run-off portion. The analyte detection electrode set may be located on a notionally separate portion of the carrier termed a reaction portion since it would be in this portion that the redox reaction is performed on the analyte. The carrier may comprise yet another portion termed the reagent portion located after the fluid sample introduction portion but before the reaction portion. The reagent portion may contain reagents diffusively bound thereto that diffuse into the fluid sample to be carried to the analyte detection electrode set to participate in the electrochemical detection and/or measurement of the analyte.

The porous carrier may take the form of a test strip. The carrier may be formed of a single piece of material or from two or more pieces of material joined together. Each piece of material may take the form of a strip. Each portion of the carrier may comprise the same material, or one or more portions may comprise a different material from the others. The function of the portion may proscribe the type of material in that portion. In all portions of the carrier, the material should be sufficiently porous for the fluid sample to permeate through the carrier by capillary action.

The sample introduction portion of the carrier may be made from any bibulous, porous or fibrous material capable of absorbing liquid rapidly. The porosity of the material can be unidirectional (i.e. with pores or fibers running wholly or predominantly parallel to an axis of the carrier) or multidirectional (e.g. omnidirectional, so that the sample introduction portion has an amorphous sponge-like structure). The material may be, for example, a porous plastic material or a cellulosic material. Porous plastic materials include, for example, polypropylene, polyethylene (preferably of very high molecular weight), polyvinylidene flouride, ethylene vinylacetate, acrylonitrile or polytetrafluoro-ethylene. Cellulosic material includes, for example, cellulose, paper, nitrocellulose or other functionalized celluloses. It may be advantageous to pre-treat the sample introduction portion with a surface-active agent during manufacture as this can reduce inherent hydrophobicity and enhance the ability to take up and deliver a moist sample rapidly and efficiently. Preferably the material should be chosen such that the sample introduction portion may be saturated with fluid, especially aqueous fluid, within a matter of seconds. Preferably the material remains robust when moist.

The reagent portion of the carrier, when present, may be made from any bibulous, porous or fibrous material capable of diffusively binding whatever reagents are to be used in the electrochemical detection and/or measurement. The material should permit free permeation of the fluid sample and be sufficiently adsorptive to permit adsorption of the reagents while permitting re-diffusion of the reagents into the fluid sample as the fluid passes through the reagent portion. Fibrous materials are particularly preferred, for example glass and fiberglass, particularly glass. Reagents diffusively bound in the reagent portion may include, for example, salts necessary for the redox reaction of the analyte, analyte conjugates for a competitive assay design, secondary antibodies conjugated to a redox probe, etc.

The reaction portion of the carrier when a separate material from the sample introduction portion or the run-off portion may be made from any bibulous, porous or fibrous material capable of having the electrodes embedded therein, for example by sandwiching the electrodes between two pieces of material. Preferably the material is capable of having the electrically conductive material of the electrodes printed thereon. An especially preferred material for the reaction portion is nitrocellulose. No chemical treatment is required which might interfere with the specific binding activity of the analyte-specific binder. Moreover, nitrocellulose is readily available in a range of pore sizes and this facilitates the selection of a carrier material to suit particular requirements such as fluid sample flow rate.

The run-off portion of the carrier may be made from any bibulous, porous or fibrous material that has sufficient absorptive capacity to allow any unbound chemicals to wash out of the reaction zone. Some examples include cellulosic materials, for example, cellulose, paper, nitrocellulose and other functionalized celluloses.

The surface of the porous carrier may be sealed, except at the sample introduction portion so that premature inclusion of moisture and inclusion of contaminants into the carrier may be prevented or reduced. Sealing may be accomplished by wrapping the carrier in a water-impervious cover, or by using a water-resistant adhesive. An adhesive has the added benefit of joining the portions together when the portions are made from separate material. Electrical leads for any electrodes should be allowed to protrude from the seal.

The apparatus may further comprise a control system for controlling the electrochemical detection and/or measurement. The control system may comprise a fluid detection electrode set (e.g. a pair of electrodes) located after the analyte detection electrode set, for example in the second portion of the carrier. The fluid detection electrode set after the analyte detection electrode set detects flow of the fluid sample in the second portion, which is an indication that the fluid sample has passed the analyte detection electrode set and that the time has arrived to initiate the electrochemical detection and/or measurement of the analyte. The fluid detection electrode set may be electrically connected to a controller, which starts the electrochemical detection and/or measurement of the analyte at the analyte detection electrode set when an appropriate signal is received from the fluid detection electrode set.

The control system may further comprise another fluid detection electrode set (e.g. a pair of electrodes) located before the analyte detection electrode set, for example in the first portion of the carrier. The fluid detection electrode set located before the analyte detection electrode set may be termed the “first” fluid detection electrode set and the fluid detection electrode set located after the analyte detection electrode set may be termed the “second” fluid detection electrode set. The first fluid detection electrode set may detect the introduction of the fluid sample on the first portion. Detection of the fluid sample by the first fluid detection electrode set may start a timer, and the timer is configured to initiate the electrochemical detection and/or measurement of the analyte by the analyte detection electrode set after a pre-determined amount of time has elapsed, provided that the second fluid detection electrode set has not already initiated the electrochemical detection and/or measurement. The first fluid detection electrode set may also be electrically connected to the controller for the purpose of sending an appropriate signal to start the timer. The controller may comprise components such as a digital processor, analog/digital (A/D) converters, memory and the timer. The pre-determined amount of time and a pre-set electrochemical detection and/or measurement procedure (e.g. a pre-set voltammetry protocol) may be stored in the memory of the controller. The controller and electrodes may be powered by any suitable power source, for example one or more batteries. Results of the electrochemical detection and/or measurement procedure may be outputted to an output device, which may be, for example, a meter, a digital numerical display, a simple lighting sequence, an audio signal or the like.

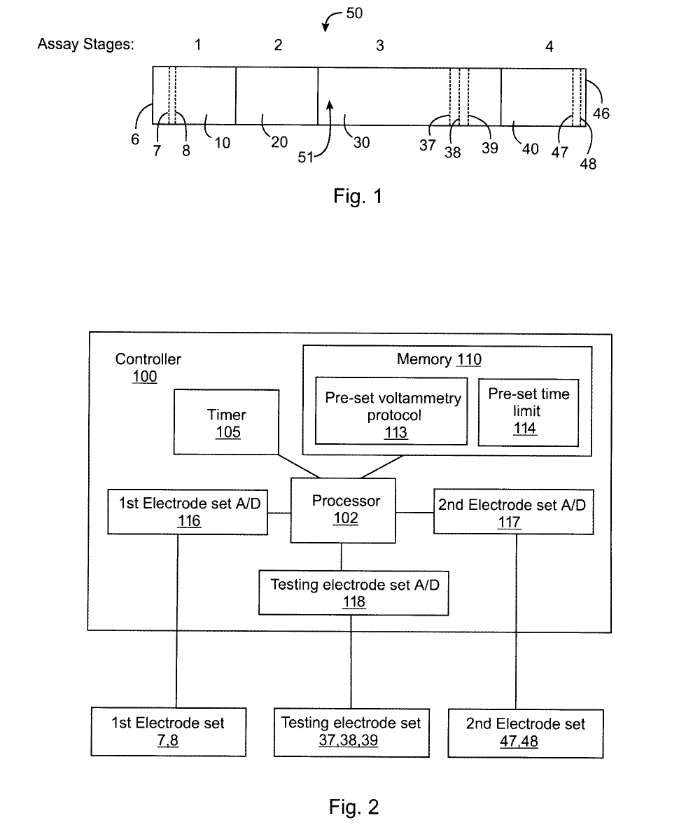

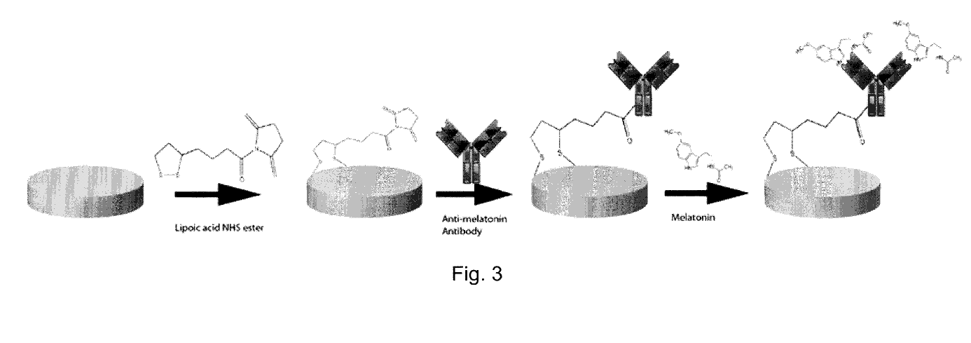

Referring to FIG. 1 and FIG. 2, a lateral flow assay apparatus 50 comprises a membrane 51 having four Assay Stages 1, 2, 3 and 4, a fluid sample flowing from Stage 1 at a proximal end 6 of the membrane 51 through to Stage 4 at a distal end 46 of the membrane 51. Stage 1 comprises a sample introduction pad 10 comprising any suitably porous material. The material for the sample introduction pad 10 should be sufficiently absorptive to permit absorption of the sample fluid, but not adsorptive such that it adsorbs the analyte from the sample fluid. Polysaccharides are particularly preferred, for example cellulose. A sample fluid (e.g. saliva) suspected of containing an analyte of interest may be introduced on to the sample introduction pad 10 where a first electrode set 7,8 comprising a first working electrode 7 and a first counter electrode 8 housed in the sample introduction pad 10 detects the introduction of the fluid sample and sends a signal to a controller 100 to start a timer 105. The signal from the first electrode set 7,8 is first converted from analog to digital by first electrode set A/D converter 116 of the controller 100 and the digital signal is then processed by a digital processor 102, which activates the timer 105. The fluid sample introduced on the sample introduction pad 10 then begins to flow across the membrane 51 by capillary action.

From the sample introduction pad 10, the fluid sample flows to Stage 2 comprising a reagent segment 20. The reagent segment 20 may comprise a fibrous material, for example glass fibers, and may contain any necessary reagents for the lateral flow assay. Such reagents may include, for example, salts, conjugates of the analyte in a competitive assay design, secondary antibodies, etc. The material for the reagent segment 20 should not be absorptive and should wick any fluid to the next stage on the lateral flow assay apparatus 50. Silicates are particularly preferred, for example asbestos and fiberglass. Any reagents may be diffusively bound (e.g. adsorbed) onto the surface of the material used in the reagent segment 20 and diffuse into the fluid sample to be carried along with the fluid sample flowing along the membrane 51.

From the reagent segment 20, the fluid sample loaded with reagents flows to Stage 3 comprising a reaction segment 30. The reaction segment 30 may comprise a porous solid phase material through which the fluid sample may flow. The material for the reaction segment 30 should be capable of carrying the sample fluid and any dissolved or dispersed that is performed on the gold electrode surface to make it specific and selective for melatonin.

X-Ray Photoelectron Spectroscopy (XPS) characterization of the electrode surface was performed on a Thermo Scientific K-Alpha spectrometer (Al-K X- ray source; 15 mA, 14 kV) before and after construction of the self-assembled monolayer. The takeoff angle between the electrode surface and the energy analyzer was 90. During analysis the operating pressure was kept at 5×10⁻¹⁰ Torr. The survey spectra (0-1100 eV) were taken on a sample analysis area of 300 μm×700 μm with an analyzer pass energy of 160 eV. The acquired data was analyzed with Thermo Scientific Avantage software and electron binding energies from the spectra scan were corrected to the Au 4f7 peak.

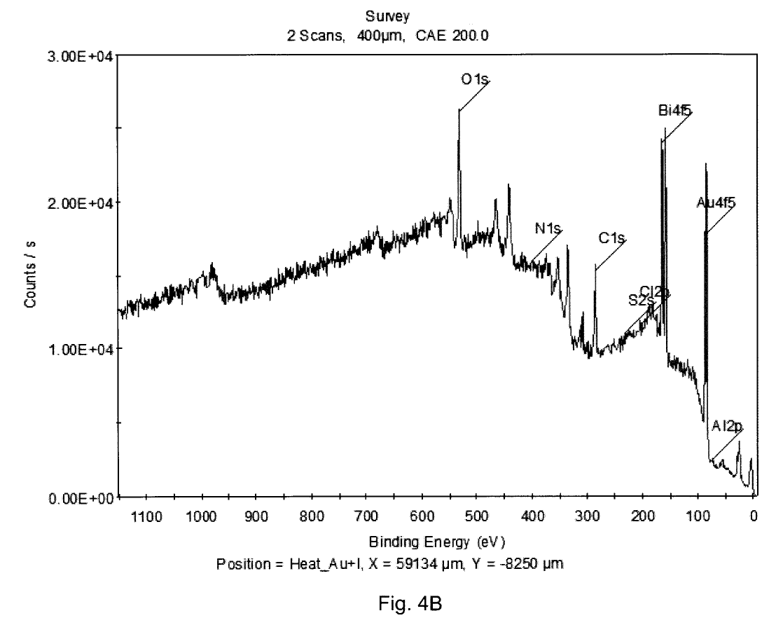

FIG. 4 depicts the XPS scans of the gold working electrode after lipoic acid (FIG. 4A) and after antibody (FIG. 4B) attachment. Increased intensity of adventitious carbon (C1s) electrons after attachment of the anti-melatonin antibody indicates that the SAM contains a highly dense layer of antibody.

Electrochemical characterization of the electrodes surface was also done. All electrochemical studies were performed at room temperature in a grounded, enclosed Faraday cage with a potentiostat/galvanostat (CompactStat, Ivium Technologies USA, Fernandina Beach, Fla.) connected to a personal computer. The screen-printed electrode was connected to the potentiostat with a three-electric contacts edge connector (Dropsens, Oviedo, Spain). Cyclic voltammetry (CV) and square wave voltammetry (SWV) were commenced from rest potential in a 10 mM Tris buffer (pH 7). The CV experiments were performed with a scan rate of 100 mVs⁻¹ in the potential range from −100 to 350 mV with a step potential of 5 mV, amplitude of 25 mV, and a frequency of 10 Hz. Electrochemical impedance spectroscopy (EIS) was performed in a 10 mM Tris buffer (pH 7) containing 5 mM sodium perchlorate and 1.0 mM ferro-/ferricyanide [Fe(CN)₆^{3-/4-} as a redox probe. The EIS measurements were conducted in the frequency range of 100 hHz to 0.1 Hz, at a formal potential of 100 mV and AC amplitude of 5 mV. The aforementioned electrochemical measurements were all repeated at least three times with separate electrodes to ensure reproducibility.

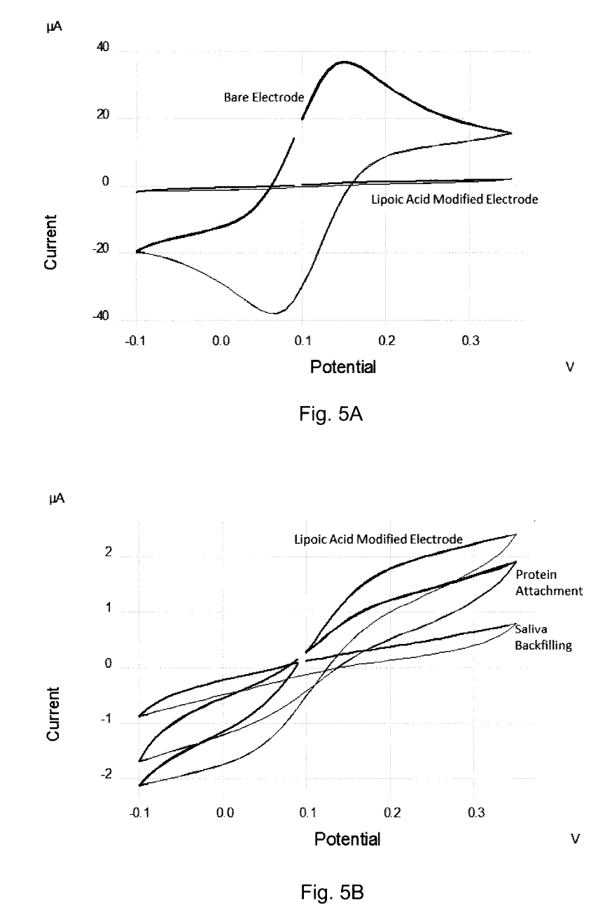

The CV scans of the bare electrode displayed large redox currents corresponding to oxidation and reduction of potential Fe(CN)₆^{3-/4-} The large redox currents shown in the voltammogram of the bare electrode surface indicates that the expected redox reactions occurred easily and quasi-reversibly on the bare gold (FIG. 5A). After each electrode modification step, the CV scan displayed charging current but did not show Faradaic signal (FIG. 5A and FIG. 5B). Furthermore, the charging current was reduced after each step. The Nyquist plots of the EIS corroborate the CV data. Impedance is presented as the sum of the real and imaginary Z components, Zᵣₑ₋ and Zᵢₘ₋, which mainly originate from the resistance and capacitance of the cell respectively. An equivalent circuit was selected to reflect the electrochemical process in order to fit accurate values.

### Example 2: Detection of Melatonin

Well known as a powerful antioxidant, melatonin is known to be oxidized at approximately 700 mV, which permits electrochemical detection of the molecule at an electrode interface. However, unlike other antioxidants, melatonin does not undergo redox cycling; upon oxidation, melatonin becomes highly reactive and frequently binds to a second melatonin molecule. The disadvantage of this is that a melatonin solution is immediately compromised after one round of oxidation and electrochemical detection must be achieved on the first round of a potential sweep of the electrode surface. However, as this example will show, if sufficient melatonin is present on the electrode surface, the electric current that results from melatonin oxidation correlates extremely well with the concentration of the original solution.

This example describes direct detection of melatonin on the surface of a gold electrode following modification with an anti-melatonin antibody to form a self-assembled monolayer (SAM). Aqueous melatonin solutions of decreasing concentration are utilized.

Experimental work with saliva spiked with known concentrations of melatonin is performed to show that saliva components have no effect on the electrochemical quantification of melatonin. Melatonin binding at the electrode surface is achieved with a melatonin receptor or antibody with a binding pocket that is specific for melatonin. This example utilizes a commercially available anti-melatonin antibody that has been shown by the manufacturer to bind only melatonin and not related tryptophan-like molecules.

An antibody-modified electrode was constructed by the procedure described in Example 1 using polyclonal anti-melatonin antibody obtained from Pierce Biotechnology Inc. (Rockford, Ill.) as the analyte-specific binder. Melatonin standard was obtained from Biotrend Chemikalien GmbH (Destin, Fla.). All other reagents and methods mentioned in this example were obtained or performed as indicated in Example 1.

### Aqueous Melatonin Detection and Measurement

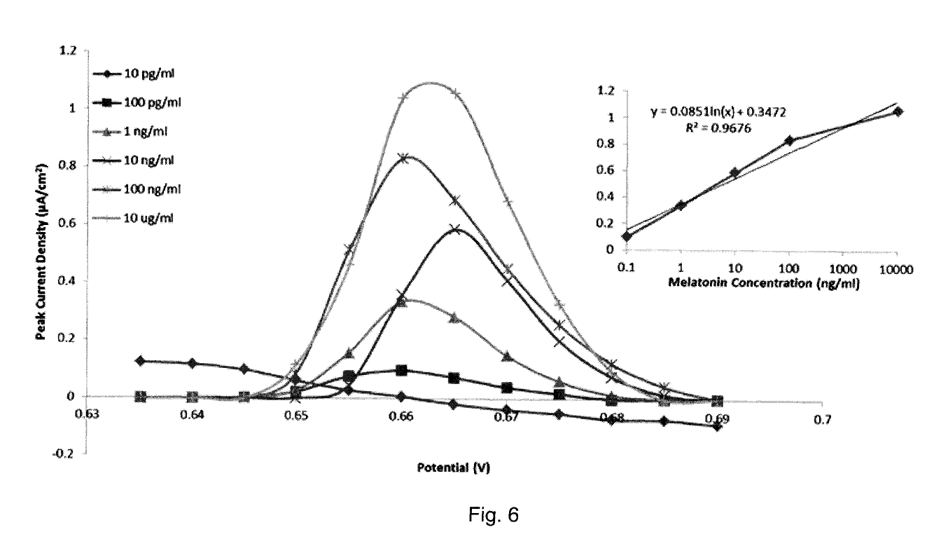

A stock solution of melatonin standard (62.5 μg/ml) was prepared in Tris buffer (pH 7.0) containing 100 mM NaCl. The stock solution was then serially diluted for melatonin measurements. For melatonin detection and measurement experiments, an 80 μL drop of the melatonin solution was deposited on the electrode for measurement such that the meniscus of the drop covered the entire surface of each electrode (working, reference, and counter). Electrochemical measurements were performed immediately. Square-wave voltammetry was performed on separate electrodes for each experiment (FIG. 6). The resulting curve was baseline corrected using IviumSoft™ (Ivium Technologies, Eindhoven, Netherlands). The peak current between the potential range of 0.6 V to 0.7 V was then obtained from the table of current vs. potential. The correlation coefficient (R²) of melatonin concentration vs. peak current was 0.9676.

### Salivary Melatonin Detection and Measurement

A stock solution of melatonin standard (1 ng/ml) was prepared in Milli-Q water. The stock solution was then serially diluted for with pooled human saliva to obtain melatonin-spiked saliva. For melatonin detection and measurement experiments, an 80 μL drop of a known concentration of melatonin-spiked saliva solution was deposited on the antibody-modified gold electrode and incubated for 1 hour in the dark at room temperature. After incubation, the electrode surface was rinsed in Milli-Q water and dried. Square-wave voltammetry measurements were performed immediately with Tris buffer containing the redox probe. The resulting curve was baseline corrected using IviumSoft™ (Ivium Technologies, Eindhoven, Netherlands). The peak current between the potential range of 0.5 V to 0.6 V was then obtained from the table of current vs. potential. The correlation coefficient (R²) of melatonin concentration vs. peak current was 0.9936.

Salivary melatonin was quantified electrochemically on an electrode that was engineered to be specific for melatonin binding and detection. The electrode permits portable, rapid, and highly sensitive quantification of endogenous salivary melatonin, and may be used in test strip devices as described in Example 3. The results show that the biosensors are capable of melatonin quantification with a limit of detection of 15 pg/ml.

### Example 3: Apparatus Construction

In this example, a lateral flow assay test strip apparatus was constructed and tested to demonstrate that electrochemical (voltammetric) detection of an analyte can be accomplished with thin-wire electrodes embedded in a nitrocellulose stage of the lateral flow assay apparatus.

Sample sheets of cellulose, glass fiber, and nitrocellulose were obtained from EMD Millipore (Billerica, Mass., USA; millipore.com). Gold wire (0.025 mm diameter), platinum wire (0.025 mm), and silver wire (0.05 mm) were obtained from Alfa Aesar (Ward Hill, Mass., USA; alfa.com). Copper wire (0.003 mm) was obtained from Omega Engineering (Laval, QC; omega.ca). Tris buffer, sodium perchlorate, ferricyanide, and ferrocyanide were obtained from Sigma Aldrich (Oakville, ON; sigmaaldrich .com).

Copper wire was cut and two pieces, each 3 cm long, were placed at each end of a laminated cardboard backing to form first and second electrode pairs, one pair at each end of the apparatus. Square pieces (0.7 mm²) of cellulose and glass fiber were then cut from larger sheets. First and second cellulose squares were placed at the ends of the laminated cardboard backing with the copper wires extending beyond the square cellulose pieces at each end of the test strip. The glass fiber square was placed on the cardboard backing next to the first cellulose square. Two strips of nitrocellulose (0.7×25 mm) were cut and placed, sandwiched together, on the available area of the cardboard backing such that the glass fiber square was located between the first cellulose square and the nitrocellulose strips. Microelectrodes constructed from the gold, platinum and silver wires (the Testing Electrode Set) were placed between the nitrocellulose strips. Acrylic, dissolved in acetone, was used to seal the sides of the test strip and hold the electrodes in place.

To determine the effectiveness of the apparatus design, electrochemical studies were performed at room temperature with an electrochemical analyzer (CH Instruments 660B, TX, U.S.A) connected to a personal computer. The Testing Electrode Set electrode was connected to the electrochemical analyzer. Testing commenced by depositing a solution of 10 mM Tris buffer (pH 7) containing 5 mM sodium perchlorate and 1.0 mM ferro-/ferricyanide [Fe(CN)₆]^{3-/4-} onto the first cellulose pad (Assay Stage 1). A multimeter was connected to the first copper electrode pair located beneath the first cellulose pad. As the solution was absorbed by the cellulose, the circuit resistance between the first electrode pair dropped to <10Ω. The multimeter was then disconnected from the first copper electrode pair and connected to the second copper electrode pair (Assay Stage 4). As the solution saturated the second cellulose pad (Assay Stage 4), the circuit resistance between the second electrode pair also dropped to <10Ω. Subsequently, a potential scan of the Testing Electrode Set was commenced from the rest potential (−2 mV) to 600 mV, with a scan rate of 100 mVs⁻¹.

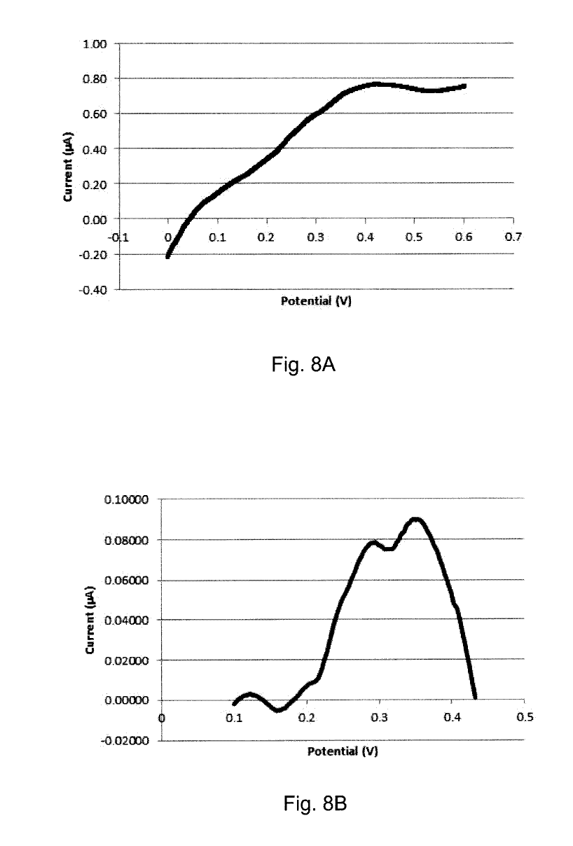

The resulting plot of current vs. potential (see FIG. 8A) clearly showed a Faradaic current wave corresponding to ferrocyanide oxidation to ferricyanide with peak current corresponding to a 0.42 V electrode potential. Subtraction of non-Faradaic current, caused by the electrolyte accepting charge from the electrode, results in a distinct faradaic wave from ferrocyanide oxidation, with a peak current of 0.09 μA (FIG. 8B). The results show that the apparatus is capable of detecting a small analyte in a solution by measuring the current associated with analyte oxidation.

This test strip device whereby thin electrochemical testing electrodes are arranged across the width of the test strip and embedded inside the strip is particularly useful when the working electrode is an electrode such as the one described in Example 1. The electrode as described in Example 1 may be printed on to the nitrocellulose rather than on a ceramic. Coupled with electrochemical detection of fluid sample introduction before the testing stage and electrochemical detection of fluid sample run-off after the testing stage, an electrochemical (e.g. voltammetric) testing protocol may be applied at an appropriate time at the testing stage to detect and measure analyte in the testing stage. The test strip device described herein offers a fast and simple solution for detecting and measuring analytes in a fluid sample.

References: The contents of the entirety of each of which are incorporated by this reference.
Allen M P. (1996) Disposable Electronic Assay Device. U.S. Pat. No. 5,580,794 issued Dec. 3, 1996.
Bekheit G E. (2000) Determination of melatonin by differential pulse voltammetry at modified carbon paste electrode. Asian Journal of Chemistry. 12(2), 541-547.
Braiek M, et al. (2012) An Electrochemical Immunosensor for Detection of Staphylococcus aureus Bacteria Based on Immobilization of Antibodies on Self-Assembled Monolayers-Functionalized Gold Electrode. Biosensors. 2, 417-426.
Clare B H, Etherington E J. (2012) Electromagnetic Detection of Analytes. United States Patent Publication US 2012-228155 published Sep. 13, 2012.
Gooding J J, Liu G. (2012) Electrochemical Sensor. U.S. Pat. No. 8,110,079 issued Feb. 7, 2012.
Hiroshi T, et al. (2005) Electrode Immobilized with Thyroid Hormone Receptor, Manufacturing Method Therefor, and Applied Use Thereof. Abstract of Japanese Patent Publication JP 2005-249725 published Sep. 15, 2009.
Hutchinson M. (2007) Method of Processing Assay Test Results. U.S. Pat. No. 7,214,542 issued May 8, 2007.
Khan M Z H, et al. (2011) Potentiometric Detection of Serotonin, Melatonin, and Their Precursors/Metabolites with Monolayer-Modified Indium Tin Oxide Electrode and Their Concentration Dependency. Sensor Letters. 9(5), 1849-1852.
Levent A. (2012) Electrochemical determination of melatonin hormone using a boron-doped diamond electrode. Diamond and Related Materials. 21, 114-119.
Lu F, et al. (2004) System for Electrochemical Quantitative Analysis of Analytes within a Solid Phase. U.S. Pat. No. 6,713,308 issued Mar. 30, 2004.
May K, et al. (1988) Immunoassays and Devices Therefor. International Patent Publication WO 88-08534 published Nov. 3, 1988.
Nazareth AR, Li S. (2013) Diagnostic Detection Device. United States Patent Publication US 2013-164858 published Jun. 27, 2013.
O'Daly J P, et al. (1995) Electrochemical Immunoassay Methods. U.S. Pat. No. 5,391,272 issued Feb. 21, 1995.
Qu W Y, et al. (2005) Electrocatalytic properties and voltammetric determination of melatonin at a nanostructured film electrode. Microchimica Acta. 150(2), 109-114.
Sasaki H, Hutchinson M. (2009) System and Method for Reading a Test Strip. U.S. Pat. No. 7,499,170 issued Mar. 3, 2009.
Zanarini S, et al. (2008) Synthesis and Electrochemiluminescence of a Ru(bpy)₃-Labeled Coupling Adduct Produced on a Self-Assembled Monolayer. J. Phys. Chem. C. 112, 2949-2957.
Zin B, Hutchinson M. (2007) Assay Test Device and Method of Making Same. U.S. Pat. No. 7,220,597 issued May 22, 2007.

The novel features will become apparent to those of skill in the art upon examination of the description. It should be understood, however, that the scope of the claims should not be limited by the embodiments, but should be given the broadest interpretation consistent with the wording of the claims and the specification as a whole.

1. An apparatus for electrochemically detecting and/or measuring an analyte in a fluid sample, the apparatus comprising:
a dry porous carrier that permits flow of a fluid sample from a first portion of the carrier to a second portion of the carrier; and, an analyte detection electrode set comprising a working electrode and a counter electrode disposed across the carrier in a non-parallel direction in relation to a direction of fluid flow such that the fluid sample flowing from the first portion of the carrier to the second portion of the carrier crosses at least the working electrode, at least the working electrode embedded in the carrier, the working electrode comprising an analyte-specific binder immobilized on an electrically conductive material, the analyte detection electrode set configured to electrochemically detect a presence of and/or measure an amount of the analyte bound to the analyte-specific binder immobilized on the working electrode after the fluid sample crosses the working electrode.

2. The apparatus according to claim 1, wherein the analyte detection electrode set is embedded in the carrier, and wherein the working and counter electrodes comprise wires or strips disposed across an entire width of the carrier perpendicular to the direction of fluid flow.

3. The apparatus according to claim 1, further comprising a first fluid detection electrode set disposed at the first portion of the carrier for detecting introduction of the fluid sample on the first portion, the first fluid detection electrode set is configured to start a timer upon detection of the fluid sample, and the timer is configured to initiate the electrochemical detection and/or measurement of the analyte by the analyte detection electrode set after a pre-determined amount of time has elapsed.

4. (canceled)

5. The apparatus according to claim 1, further comprising a second fluid detection electrode set disposed at the second portion of the carrier for detecting flow of the fluid sample in the second portion, the second fluid detection electrode set configured to initiate the electrochemical detection and/or measurement of the analyte by the analyte detection electrode set when the second fluid detection electrode set detects the fluid sample.

6. The apparatus according to claim 1, wherein the electrochemical detection and/or measurement of the analyte is accomplished by a pre-determined cyclic voltammetry protocol.

7. (canceled)

8. The apparatus according to claim 1, wherein the porous carrier comprises a test strip having a fluid sample introduction portion, a reagent portion, a reaction portion and a run-off portion, the first portion corresponding to the fluid sample introduction portion and the second portion corresponding to the run-off portion, the reaction portion containing the analyte detection electrode set, and the reagent portion containing reagents diffusively bound thereto that diffuse into the fluid sample to be carried to the reaction portion to participate in the electrochemical detection and/or measurement of the analyte.

9. The apparatus according to claim 8, wherein the reaction portion comprises nitrocellulose, the sample introduction portion comprises cellulose, the reagent portion comprises glass fiber and the run-off portion comprises cellulose.

10. (canceled)

11. The apparatus according to claim 1, wherein the analyte-specific binder is linked to the electrically conductive material by a bifunctional linker, the bifunctional linker comprising dihydrolipoic acid N-hydroxysuccinimide ester.

12. (canceled)

13. (canceled)

14. The apparatus according to claim 1, wherein the analyte-specific binder forms a self-assembled monolayer on the electrically conductive material and gaps in the self-assembled monolayer on the electrically conductive material are filled with an analyte-free fluid comprising human pooled saliva or polyethylene glycol.

15. (canceled)

16. (canceled)

17. The apparatus according to claim 1, wherein the electrically conductive material of the working electrode comprises gold and the analyte-specific binder comprises an antibody.

18. (canceled)

19. (canceled)

20. The apparatus according to claim 1, wherein the analyte detection electrode set further comprises a reference electrode.

21. (canceled)

22. The apparatus according to claim 1, wherein the analyte-specific binder is specific for melatonin and the fluid sample comprises saliva, blood or urine.

23. (canceled)

24. A biosensor for detecting and/or measuring melatonin in a fluid sample, the biosensor comprising an electrochemical device comprising:
a working electrode with a self-assembled monolayer of a melatonin-specific antibody immobilized thereon, the antibody attached to the working electrode by a bifunctional linker; and,
a counter electrode.

25. The biosensor according to claim 24, wherein the bifunctional linker comprises dihydrolipoic acid N-hydroxysuccinimide ester and the working electrode comprises gold.

26. The biosensor according to claim 24, wherein gaps in the self-assembled monolayer on the working electrode are filled with a melatonin-free fluid comprising human pooled saliva or polyethylene glycol.

27. (canceled)

28. (canceled)

29. The biosensor according to claim 24, wherein the counter electrode comprises platinum, the antibody is a polyclonal antibody, the electrochemical device further comprises a reference electrode comprising silver and the electrodes are printed on a substrate comprising nitrocellulose and the electrodes are sandwiched between nitrocellulose strips.

30. (canceled)

31. (canceled)

32. (canceled)

33. (canceled)

34. A method of electrochemically detecting and/or measuring an analyte in a fluid sample, the method comprising:
(a) contacting a fluid sample with a dry porous carrier to permit flow of the fluid sample in the carrier by capillary action;
(b) permitting the fluid sample to contact an analyte detection electrode set comprising a working electrode and a counter electrode embedded in the carrier, the working electrode comprising an analyte-specific binder immobilized on an electrically conductive material, the analyte-specific binder binding analyte present in the fluid sample thereby immobilizing the analyte at the working electrode; and,
(c) permitting the fluid sample to flow past the analyte detection electrode set, detecting the fluid sample with a fluid detection electrode set after the fluid sample has passed the analyte detection electrode set and initiating electrochemical detection and/or measurement of the analyte by the analyte detection electrode set when the fluid detection electrode set detects the fluid sample.

35. The method according to claim 34, wherein the electrochemical detection and/or measurement comprises a pre-set cyclic voltammetry protocol.

36. (canceled)

37. The method according to claim 34, wherein the fluid detection electrode set that detects the fluid sample after the analyte detection electrode set is a second fluid detection electrode set, and wherein the method further comprises detecting the fluid sample before the fluid sample contacts the analyte detection electrode set and starting a timer upon detection of the fluid sample before the fluid sample contacts the analyte detection electrode set to initiate the electrochemical detection and/or measurement of the analyte by the analyte detection electrode set after a pre-determined amount of time has elapsed if the electrochemical detection and/or measurement has not been initiated before the pre-determined amount of time by the second fluid detection electrode set.

38. The method according to claim 34, wherein the fluid sample comprises saliva and the analyte is melatonin.

39. (canceled)
